(19) United States
(12) Patent Application Publication
(10) Pub. No.: US 2005/0278022 A1
(43) Pub. Date: Dec. 15, 2005

(54) Annuloplasty prostheses with improved anchoring structures, and related methods

(75) Inventor: Jyue Boon Lim, Minneapolis, MN (US)

Correspondence Address: FISH & NEAVE IP GROUP;ROPES & GRAY LLP, 1251 AVENUE OF THE AMERICAS FL C3, NEW YORK, NY, 10020-1105, US

(73) Assignee: St. Jude Medical, Inc.

(21) Appl. No.: 11/146,873

(22) Filed: Jun. 6, 2005

Related U.S. Application Data
(60) Provisional application No. 60/579,737, filed on Jun. 14, 2004.

(51) Int. Cl. A61F 2/24

(52) U.S. Cl. 623/2.36; 623/902

(57) ABSTRACT

An annuloplasty prosthesis is less than a complete ring (e.g., it may be C-shaped or U-shaped). The prosthesis has a structural member that basically gives the prosthesis its shape. The structural member is provided with surface portions that are transverse to adjacent portions of the surface of the structural member. At least two of these transverse surface portions are spaced from one another and face toward one another along the length of the structural member. Sutures that are used to implant the prosthesis and that are respectively adjacent to these two transverse surface portions are thereby prevented from moving farther apart along the length of the prosthesis.

This application claims the benefit of U.S. provisional patent application 60/579,737, filed Jun. 14, 2004, which is hereby incorporated by reference herein in its entirety.

### BACKGROUND OF THE INVENTION

Annuloplasty prostheses that are less than completely annular are well known as is shown, for example, by Carpentier U.S. Pat. No. 3,656,185. Because such a prosthesis is less than a full ring, it can be difficult to implant the prosthesis so that the tissue to which it is secured cannot spread (or continue to spread) along the length of the prosthesis. A structural member of the prosthesis may be covered with a soft fabric cover. The prosthesis may be sutured into the patient by sutures that pass through the fabric cover and also through adjacent tissue. However, the fabric cover may not be strong enough to resist stretching or to prevent the sutures from tearing out of the fabric, especially near one or both ends of the prosthesis; in which cases the tissue may be able to move (or continue to move) relative to the prosthesis, e.g., by spreading along the length of the prosthesis. Because it is often an objective of the prosthesis to reverse or prevent such tissue movement, the prosthesis may be less effective than desired.

### SUMMARY OF THE INVENTION

An annuloplasty prosthesis in accordance with the invention is less than a full ring. It is, however, curved to follow a portion of the annulus of a heart valve such as a mitral or tricuspid valve. For example, the prosthesis may be C shaped or U shaped. The prosthesis includes an elongated structural member that basically gives the prosthesis its shape. This structural member has at least two surface portions that are transverse to adjacent portions of the surface of the structural member. These two transverse surface portions are spaced from one another along the length of the structural member. They also face toward one another along the length of that member. When the prosthesis is sutured into a patient, tissue adjacent to each of the transverse surface portions is sutured to the prosthesis by sutures that pass adjacent those transverse surface portions. The presence of the transverse surface portions prevents these sutures and the tissue engaged by these sutures from moving away from one another along the length of the prosthesis.

Further features of the invention, its nature and various advantages, will be more apparent from the accompanying drawings and the following detailed description.

### BRIEF DESCRIPTION OF THE DRAWINGS

FIG. 1 is a simplified perspective view of an illustrative embodiment of an annuloplasty prosthesis in accordance with the invention.

FIG. 2 is a simplified perspective view of another illustrative embodiment of an annuloplasty prosthesis in accordance with the invention.

FIG. 3 is a simplified perspective view of a portion of yet another illustrative embodiment of an annuloplasty prosthesis in accordance with the invention.

FIG. 4 is a simplified perspective view of a portion of still another illustrative embodiment of an annuloplasty prosthesis in accordance with the invention.

FIG. 5 is a simplified perspective view of a portion of yet another illustrative embodiment of an annuloplasty prosthesis in accordance with the invention.

FIG. 6 is a simplified perspective view of yet another illustrative embodiment of the invention.

FIG. 7 is a simplified perspective view of still another illustrative embodiment of the invention.

FIG. 8 is a simplified perspective view of an illustrative prosthesis in use in a patient in accordance with the invention.

FIG. 9 is a simplified enlargement of a representative portion of an illustrative embodiment in accordance with the invention.

FIG. 10 is a simplified sectional view of a representative portion of another illustrative embodiment of the invention.

FIG. 11 is a simplified sectional view of a representative portion of another illustrative embodiment of the invention.

### DETAILED DESCRIPTION

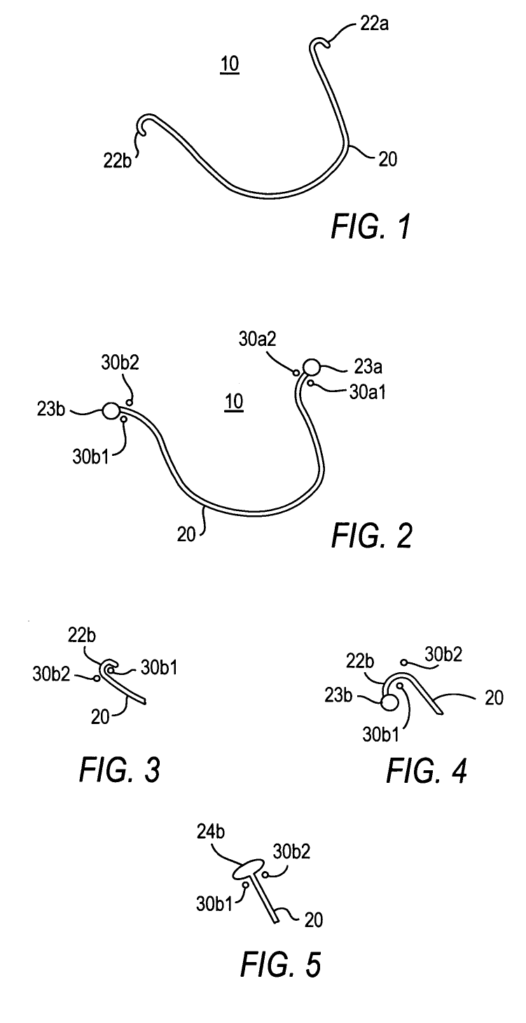

As is shown in FIG. 1, an illustrative embodiment of an annuloplasty prosthesis 10 in accordance with the invention includes a structural member 20 that is longitudinal but curved along its length. In addition, structural member 20 has attachment hooks 22a and 22b at respective opposite ends of its length. Structural member 20 is preferably substantially rigid or semi-rigid. At the very least, structural member has sufficient rigidity to cause prosthesis 10 to generally hold a predetermined shape, although perhaps with some flexibility (i.e., ability to move or flex with the tissue to which the prosthesis has been sutured when it is implanted in a patient). A rigid prosthesis exhibits little or no movement or flexing in response to attempted normal movement of adjacent tissue. A semi-rigid prosthesis exhibits more movement or flexing in response to attempted normal movement of adjacent tissue. Prostheses in accordance with this invention may be either rigid or semi-rigid in these terms.

Prosthesis 10 is less than a complete ring or annulus. It is, however, curved to follow or correspond to a portion of the annulus of a heart valve such as a mitral valve or a triscuspid valve. In the case of a mitral valve, for example, the main portion of structural member 20 may be curved to follow the posterior portion of the mitral valve annulus, with attachment hook 22a adjacent one trigone of the valve and attachment hook 22b adjacent the other trigone of the valve. As a general matter, structural member 20 has an overall U or C shape.

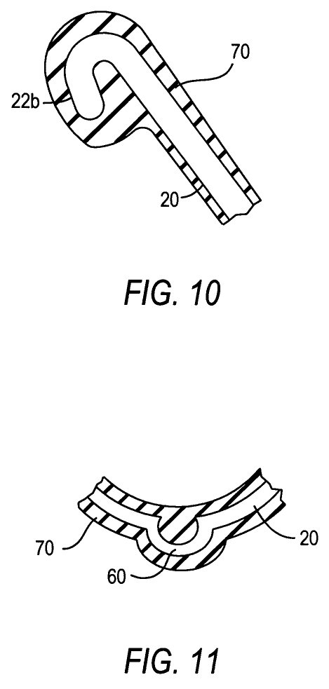

Structural member 20 may be covered with a soft fabric cover (not shown in FIG. 1, but shown at 50 in FIG. 9). Although this fabric cover may even cover attachment hooks 22a and 22b, it preferably leaves the presence of these hooks visibly evident to a surgeon implanting the prosthesis. Other cover or buffering materials may be used instead of or in addition to fabric. For example, FIGS. 10 and 11 show use of silicone or other generally similar, soft, polymeric material as a substantially continuous coating, cover, or buffering layer 70 over structural member 20. Note that although in FIGS. 10 and 11 cover 70 has been cut away to reveal structural member 20 inside the cover, cover 70 actually completely covers the features of structural member 20 like attachment hook 22b or loop 60. If desired, cover material like 70 may be further covered with fabric like 50 (FIG. 9). At least some of the cover material 50 and/or 70 that is used is preferably penetrable by a suture needle and associated suture material. It will be understood, however, that such penetrability of any cover 50/70 over structural member 20 is not a requirement for all embodiments of the invention.

Structural member 20 may be basically flat (i.e., basically two-dimensional and therefore basically lying in an x-y plane), or structural member 20 may have a more complex three-dimensional shape such as a basically saddle shape. By saddle shape it is meant that if one were to look down on the prosthesis, structural member 20 would curve upwardly for some distance as one moved away from each of hooks 22a and 22b along the length of member 20. Thereafter, however, the curvature of structural member 20 would reverse. In other words, adjacent to hooks 22a and 22b, the z-axis radii of curvature extend upwardly relative to an x-y plane; but where the curvature reverses, the z-axis radii of curvature extend downwardly relative to the x-y plane. All curvature transitions are preferably smooth. The highest point is preferably about midway between hooks 22a and 22b. From above, the prosthesis is still basically C- or U-shaped. Such a saddle shape may better conform to a saddle-shaped heart valve annulus.

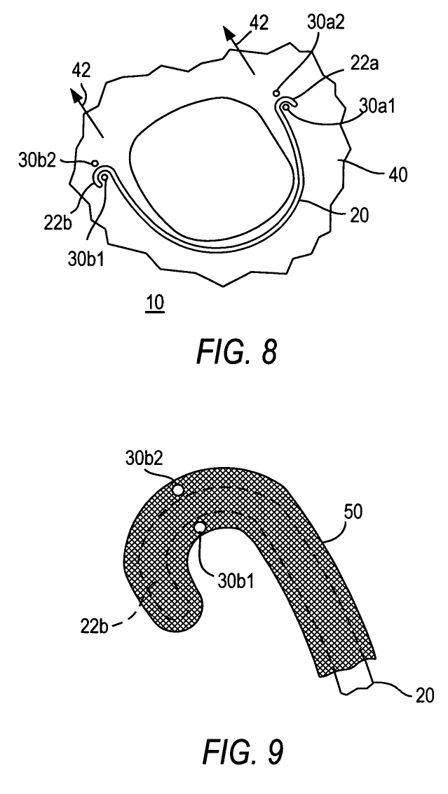

Attachment hooks 22a and 22b extend transversely to the length (or surface) of the rest of structural member 20 adjacent to those hooks. Attachment hooks 22a and 22b open toward one another along the length of structural member 20. Attachment hooks 22a and 22b are preferably prominent enough in the transverse direction (i.e., they extend far enough out from the adjacent surface of structural member 20) to securely engage sutures that are passed through those hooks into adjacent tissue when prosthesis 10 is implanted. For example, FIG. 8 shows prosthesis 10 implanted in a patient by being sutured to cardiac tissue 40 of the patient. One suture loop, which includes legs 30a1 and 30a2, is passed through attachment hook 22a and the adjacent portion of tissue 40. In other words, suture loop 30a1/30a2 passes inside hook 22a (leg 30a1), through some tissue 40 below that hook, outside hook 22a (leg 30a2), and may then be tied together above the hook. Suture loop 30b1/30b2 is similar with respect to attachment hook 22b (i.e., leg 30b1 passes inside hook 22b, the suture continues through tissue below the hook, leg 30b2 passes outside hook 22b, and the suture is tied off above hook 22b). Additional suture loops (not shown) may be used at other points along the length of prosthesis 10 between hooks 22a and 22b. FIG. 9 shows that structural member 20 may be covered by fabric cover 50, and that suture loop legs like 30b1 and 30b2 may pass through that fabric.

By having sutures inside hooks 22a and 22b (especially suture loop legs 30a1 and 30b1), the tissue thus secured to prosthesis 10 is prevented from moving away from the ends of prosthesis 10 (e.g., along the length of the prosthesis (i.e., in the directions and at the locations indicated by arrows 42 in FIG. 8)). This helps to keep the length of the portion of the valve annulus that follows prosthesis 10 from getting larger than the length of the prosthesis between hooks 22a and 22b. This is a very desirable attribute for a prosthesis of this type. The securement of tissue adjacent to hooks 22a and 22b is very firm and positive because features of structural member 20 (i.e., the hooks), in cooperation with sutures 30, directly oppose motion of tissue relative to prosthesis 10 in directions and at locations 42. In particular, the surfaces of hooks 22a and 22b that are transverse to the adjacent structural member surfaces or length and that are at the bottom of each hook, in cooperation with the suture loop legs 30a1 and 30b1 that are in the hooks, make it substantially impossible for suture loops 30 to move beyond the ends of prosthesis 10. This is the result of direct opposition of two essentially structural members (hooks 22, on one hand, and sutures 30, on the other hand). It does not rely on mere frictional resistance, such as a suture tied around a smooth, longitudinal, structural member of the prosthesis. Nor does it rely on the tear-strength of a fabric cover 50 (and/or other cover 70) on the prosthesis, such as when the suture merely passes through the cover of an otherwise smooth structural member of the prosthesis.

The invention is equally effective whether suture loop legs 30a1 and 30b1 contact hooks 22a and 22b directly, or engage the hooks less directly through intervening cover material 50 and/or 70. Both of these possibilities will be understood to be covered by references herein to contact or engagement between sutures and transverse surface features of a prosthesis structural member (e.g., hooks 22).

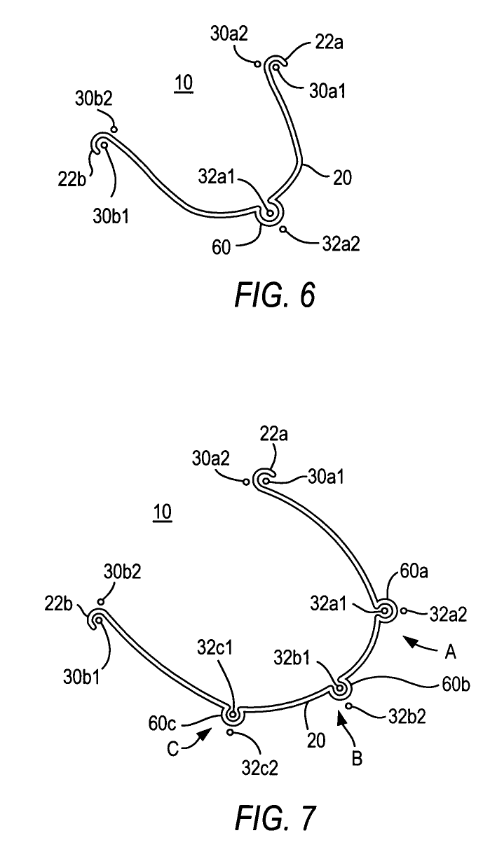

Again, to ensure good and secure engagement between hooks 22 and sutures 30, the interior of each hook is preferably at least as large as (preferably somewhat larger than) the cross-sectional diameter of suture material 30. The same is true for the transverse prominence of all other types of transverse surface features shown in subsequent FIGS. For example, the portion of the radius of a ball 23 in FIG. 2 that extends traversely beyond the adjacent surface of member 20 is preferably at least as large as the diameter of suture material 30. The transverse extension or projection of an arm of T 24 in FIG. 5 is preferably at least as large as the diameter of suture material 30. And the interior of any of loops 60 in FIGS. 6, 7, and 11 is preferably at least as large as the diameter of suture material 30.

FIG. 2 shows an alternative embodiment in which, instead of a hook 22, each end of structural member 20 has an enlarged ball 23a or 23b. FIG. 2 shows where the legs 30a1, 30a2, 30b1, and 30b2 of suture loops can be placed relative to the balls 23 on structural members 20. For example, suture loop 30a1/30a2 is preferably placed around structural member 20 (and through adjacent tissue below (not shown)) immediately adjacent to ball 23a. Ball 23a prevents suture loop 30a1/30b1 from coming off the adjacent end of the prosthesis. This prevents the adjacent tissue from moving relative to that portion of the prosthesis (e.g., away from that end of the prosthesis along the length of the prosthesis). Suture loop 30b1/30b2 has a similar relationship to ball 23b on the other end of structural member 20.

FIG. 3 illustrates an alternative embodiment in which representative hook 22b is turned inwardly of the C- or U-shaped structural member 20 of the prosthesis (rather than being turned outwardly as in FIG. 1). In other respects the embodiment shown in FIG. 3 can be similar to the embodiment shown in FIG. 1. FIG. 3 shows illustrative placement of the legs 30b1 and 30b2 of a representative suture loop for the purposes of this invention.

FIG. 4 shows another alternative embodiment in which the depicted representative end of prosthesis structural member 20 has both a hook 22b and an enlarged ball 23b on the free end of the hook. Hooks like 22b work like the hooks in FIG. 1 (in cooperation with the legs 30b1 and 30b2 of the depicted representative suture loop). Ball 23b prevents suture loop 30b1/30b2 from slipping off the free end of hook 22b. Ball 23 may also help to give the prosthesis a more atraumatic end. In other respects, the embodiment of FIG. 4 can be like the embodiment of FIG. 1.

FIG. 5 shows another illustrative embodiment in which the depicted representative end of prosthesis structural member 20 has a transversely enlarged T shape 24b. FIG. 5 also shows where legs 30b1 and 30b2 of the depicted representative suture loop can be placed relative to T-shaped end 24b. In particular, this suture loop can be tied around structural member 20 just inside of T-shaped end 24b, with, of course, a portion of the suture passing through adjacent tissue (not shown). T-shaped end 24b prevents suture loop 30b1/30b2 from moving off the end of structural member 20. Except for this difference in end shape, the embodiment shown in FIG. 5 can be similar to previously described embodiments.

FIG. 6 shows an alternative embodiment that is basically similar to FIG. 1, but with the addition of another transversely extending structure 60 on structural member 20 approximately midway along the length of that member between hooks 22a and 22b. Structure 60 is a transversely extending eyelet or loop (which may or may not be completely closed). FIG. 6 shows legs 32a1 and 32a2 of a suture loop that passes through loop 60 (and also through some adjacent tissue (not shown)). This arrangement helps to prevent tissue adjacent to eyelet 60 from moving along the length of structural member 20 relative to that member. This additionally helps to stabilize prosthesis 10 and the adjacent tissue relative to one another. For example, it helps to prevent elongation or enlargement of any portion of the valve annulus that is adjacent to prosthesis 10.

The embodiment shown in FIG. 7 can be similar to what is shown in FIG. 6, but with more eyelets or loops 60a, 60b, and 60c spaced along the length of structural member 20 between end hooks 22a and 22b. Each of eyelets 60a-c can be similar to eyelet 60 in FIG. 6, and each of eyelets 60a-c can have an associated suture loop 32a1/32a2, 32b1/32b2, 32c1/32c2 passing through it in the same manner as is described above for suture loop 32a1/32a2 in FIG. 6. Additional eyelets 60a-c provide even more resistance to movement of tissue along the length of structural member 20.

FIGS. 10 and 11 show representative portions of other illustrative embodiments in which silicone or other soft, polymeric material is applied as a substantially continuous coating or cover 70 over structural member 20. Because in these embodiments cover 70 completely covers the transverse structural member features like hook 22b or loop 60, this cover material should be penetrable by a suture needle and suture material so that the suture material can engage the structural member transverse features in the effective manner described herein for this invention. Completely covering the structural member transverse features as in FIGS. 10 and 11 may have the advantage of increasing the size of the cover material portion through which the surgeon can sew sutures, thereby making suturing easier for the surgeon.

Recapitulating the foregoing in somewhat different terms, a rigid or semi-rigid, C-shaped, annuloplasty prosthesis with anchoring or attachment features is provided. The anchoring or attachment features may be provided at the ends of the prosthesis, along the posterior section of the prosthesis (e.g., for mitral valve use), or in both locations. The anchoring and attachment features disclosed herein securely attach the annuloplasty prosthesis to the mitral valve trigones or annulus, for example. The prosthesis may comprise any of a number of profiles (e.g., a saddle shape, a flat C-shape, an asymmetrical shape, or any other suitable shape). The annuloplasty prosthesis device may be used for mitral valve repair by supporting the posterior section of the mitral annulus and by reducing the diameter of the mitral valve annulus so that the leaflets can once again coapt properly.

FIGS. 1-11 illustrate various embodiments of a rigid or semi-rigid, C-shaped, annuloplasty prosthesis with anchoring or attachment features in accordance with the principles of the present invention. The C-prosthesis may comprise a core 20. The core may be covered in a fabric sleeve or other protective coating 50 and/or 70. The cover 50/70 may provide additional or alternative means of attaching the annuloplasty prosthesis in place. The core 20 may be comprised of any number of illustrative materials (e.g., a polymer such as ultra-high-molecular-weight polyethylene, polyurethane, ABS, etc.). These materials may allow the core to both flex and hold its shape. Shape memory alloys such as Nitinol may also be used to create such a semi-rigid prosthesis with similar desirable flexibility features.

Using anchoring and attachment features like those illustrated in FIGS. 1-11 allows the sutures to engage both the core material 20 and the covering 50/70. The hooks, rounded ends, T-shaped ends, and attachment loops illustrated in FIGS. 1-11 provide additional features to suture through or around, thereby further anchoring the prosthesis in place.

FIGS. 1-5 illustrate anchoring ends 22, 23, 24 that provide gripping features for the sutures so that core 20 may be better integrated with the valve annulus. Features of FIGS. 1 and 2 may be combined to provide an even more secure anchoring end feature such as that shown in FIG. 4. The ends can be of any shape and size suitable to provide the desirable attachment means.

These anchoring and attachment features may prevent tearing of the fabric 50 and/or other cover 70, because the core 20 of the prosthesis is also sutured to the trigones or annulus. This reduces the ability of core 20 to put stress on the polyester fabric or other covering 50 and/or 70 that is sutured to the trigones or annulus, which may cause tearing.

The attachment loop 60 shown in FIG. 6 or FIG. 11 and located at the central posterior section of the annulus (e.g., at the so-called P2 portion of the mitral valve annulus) may serve a similar purpose as the end attachment features described above. Additionally, attachment loops 60 such as those illustrated in FIGS. 6, 7, and 11 may further benefit a mitral valve annulus that continues to dilate posteriorly. The loop(s) will hold the posterior annulus in place without allowing it to dilate further. If sutures engage only the annuloplasty prosthesis's fabric or other covering, the fabric or other covering may stretch as an annulus continues to move and dilate. Thus, regurgitation may again become present during the valve's operation. The loop of FIG. 6 or FIG. 11 can also be moved to points A or C of FIG. 7 (the right lateral side of the mitral valve annulus (e.g., P3) or the left lateral side of the annulus (e.g., P1), respectively). The posterior section of the annuloplasty prosthesis may have attachment loops 60 at each of the three posterior segments A, B, and C (FIG. 7). The loops can be of any shape or size to provide the desired attachment features.

All of the features described above provide additional suture points along the annuloplasty prosthesis such that sutures may engage both the core 20 and the covering 50/70.

It will be understood that the foregoing is only illustrative of the principles of the invention, and that various modifications can be made by those skilled in the art without departing from the scope and spirit of the invention. For example, any of the embodiments can have fabric covers 50 and/or other covers 70, or not, as desired. Any suitable material(s) can be used for structural member 20 and the cover 50/70 (if included). Any number of sutures can be tied around structural member 20 along its length. If a cover 50/70 is included, the sutures can pass through that cover, or not, as desired.

1. An annuloplasty prosthesis comprising:
an elongated structural member that is curved along its length to correspond to a portion of a cardiac valve annulus, the structural member including first and second surface portions that are transverse to adjacent portions of the surface of the structural member and that are spaced from and face toward one another along the length of the structural member.

2. The prosthesis defined in claim 1 further comprising:
a cover covering the structural member.

3. The prosthesis defined in claim 2 wherein the cover comprises fabric.

4. The prosthesis defined in claim 2 wherein the cover comprises a coating of substantially continuous, polymeric material.

5. The prosthesis defined in claim 1 wherein the structural member is substantially rigid.

6. The prosthesis defined in claim 1 wherein the structural member is semi-rigid.

7. The prosthesis defined in claim 1 wherein the first and second surface portions are adjacent respective opposite ends of the structural member.

8. The prosthesis defined in claim 1 wherein the structural member further includes a third surface portion that is transverse to the adjacent portion of the surface of the structural member and that is intermediate the first and second surface portions.

9. The prosthesis defined in claim 1 wherein the first and second surface portions are each prominent enough to be engaged by suture material.

10. The prosthesis defined in claim 1 wherein at least one of the first and second surface portions comprises a hook that extends transversely from the adjacent portion of the structural member.

11. The prosthesis defined in claim 1 wherein at least one of the first and second surface portions comprises a transverse enlargement of the structural member.

12. The prosthesis defined in claim 1 wherein at least one of the first and second surface portions comprises a loop formed by the structural member.

13. The prosthesis defined in claim 1 wherein the structural member is generally U-shaped.

14. The prosthesis defined in claim 1 wherein the structural member is generally C-shaped.

15. The prosthesis defined in claim 1 wherein the structural member lies substantially in a plane.

16. The prosthesis defined in claim 1 wherein the structural member lies substantially in a saddle-shaped geometrical surface.

17. An annuloplasty method comprising:
providing an annuloplasty prosthesis that includes an elongated structural member that is curved along its length to correspond to a portion of a cardiac valve annulus, the structural member including first and second surface portions that are transverse to adjacent portions of the surface of the structural member and that are spaced from and face toward one another along the length of the structural member;
implanting the prosthesis so that the structural member is adjacent and generally parallel to the portion of the cardiac valve annulus; and
suturing the prosthesis to adjacent tissue, with at least some sutures used in the suturing being respectively adjacent the first and second surface portions so that the first and second surface portions resist movement of those sutures farther away from one another along the length of the structural member.

18. The method defined in claim 17 wherein the prosthesis further includes a suture-penetrable cover over the structural member, and wherein the suturing comprises:
passing the sutures through the cover to respectively engage the first and second surface portions.

19. The method defined in claim 17 wherein the prosthesis further includes a third surface portion that is transverse to the adjacent portion of the surface of the structural member and that is intermediate the first and second surface portions, and wherein the suturing comprises:
applying a further suture adjacent the third surface portion.

20. The method defined in claim 19 wherein the third surface portion comprises a transverse loop of the structural member, and wherein the applying comprises:
passing the further suture through the loop.
